(19) United States
(12) Patent Application Publication
(10) Pub. No.: US 2026/0216040 A1
(43) Pub. Date: Jul. 30, 2026

(54) COMPOSITIONS AND METHODS OF USE THEREOF FOR IMPROVEMENT OF SKIN HEALTH

(71) Applicant: The WellBeauty Company LLC, Tysons Corner, VA (US)

(72) Inventors: Naomi Whittel, Tysons Corner, VA (US); Rob Maru, Livingston, NJ (US); Sebastian Balcombe, Boca Raton, FL (US)

(73) Assignee: The WellBeauty Company LLC, Tysons Corner, VA (US)

(21) Appl. No.: 19/455,200

(22) Filed: Jan. 21, 2026

Related U.S. Application Data
(63) Continuation of application No. 18/961,098, filed on Nov. 26, 2024.
(60) Provisional application No. 63/616,253, filed on Dec. 29, 2023.

(51) Int. Cl. A61K 8/65; A61K 8/67; A61K 8/73; A61Q 19/08

(52) U.S. Cl. CPC A61K 8/65; A61K 8/676; A61K 8/735; A61Q 19/08

(57) ABSTRACT

Disclosed herein is a composition for improving skin health of a subject an effective amount of collagen and elastin. In certain embodiments, collagen and elastin are present at a collagen to elastin ratio of about 10:1. In certain embodiments, the disclosed composition further comprises one or more additional skincare ingredient, such as hyaluronic acid. Further disclosed herein is a method skin health of a subject comprising administering to the subject a composition disclosed herein. In certain embodiments, improving skin health produces a reduction or reversal of the appearance of skin aging in a subject. According to certain embodiments, improved skin health is measured by one or more of a reduction in periocular fine lines, periocular wrinkles, and/or global wrinkles.

### CROSS-REFERENCE TO RELATED APPLICATION(S)

This application is a continuation of U.S. application Ser. No. 18/961,098, filed Nov. 26, 2024, which claims the benefit of U.S. Provisional Application No. 63/616,253 filed Dec. 29, 2023, and entitled “Compositions of Improved Skin Care,” which is hereby incorporated by reference in its entirety under 35 U.S.C. § 119(e).

### TECHNICAL FIELD

The disclosed technology relates generally to compositions, methods, and system for improving the appearance of human skin.

### BACKGROUND

There has been a long felt need for compositions improve the health and appearance of human skin. Human skin undergoes significant changes as a person ages. The skin becomes thinner and drier due to the thinning of the epithelial layer and degeneration of the underlying fat and connective tissue. This results in a loss of skin volume, elasticity, firmness, and resiliency. The skin of the face and body areas are particularly prone to signs of aging, which can include wrinkles, fine lines, thinning skin, sagging skin, sinking skin, dryness, and itchiness. These changes can have a significant impact on a person's appearance and overall skin health. There is a need in the art for compositions and methods to reduce or reverse the effects of aging on skin.

### BRIEF SUMMARY

Disclosed herein is a composition for improving skin health of a subject an effective amount of collagen and elastin. In certain embodiments, collagen and elastin are present at a collagen to elastin ratio of about 10:1. In certain embodiments, the disclosed composition further comprises one or more additional skincare ingredients. In certain embodiments, one or more additional skincare ingredients is hyaluronic acid.

Further disclosed herein is a method skin health of a subject comprising administering to the subject a composition disclosed herein. In certain embodiments, improving skin health produces a reduction or reversal of the appearance of skin aging in a subject. According to certain embodiments, improved skin health is measured by one or more of a reduction in periocular fine lines, periocular wrinkles, and/or global wrinkles. According to certain further embodiments, improved skin health is measured by one or more of an increase in skin firmness, radiance, smoothness, pinch recoil, extensibility, resiliency, pure elasticity, and/or bio-elasticity.

While multiple embodiments are disclosed, still other embodiments of the disclosure will become apparent to those skilled in the art from the following detailed description, which shows and describes illustrative embodiments of the disclosed compositions, systems and methods. As will be realized, the disclosed compositions, systems and methods are capable of modifications in various obvious aspects, all without departing from the spirit and scope of the disclosure. Accordingly, the drawings and detailed description are to be regarded as illustrative in nature and not restrictive.

### DETAILED DESCRIPTION

Before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “subject” refers to the target of administration, e.g. a subject.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause unacceptable adverse side effects. The specific therapeutically effective dose level for any particular subject will depend upon a variety of factors including; the specific composition employed; the age, body weight, general health, sex and diet of the subject; the time of administration; the route of administration; the rate of excretion of the specific compound employed; the duration of the treatment; drugs used in combination or coincidental with the specific compound employed and like factors well known in the art. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is substantially free of particles would either completely lack particles, or so nearly completely lack particles that the effect would be the same as if it completely lacked particles. In other words, a composition that is substantially free of an ingredient or element may still actually contain such item as long as there is no measurable effect thereof.

As used herein, the term “synergistic effect” or grammatical variations thereof means and includes a cooperative action encountered in a combination of two or more active compounds in which the combined activity of the two or more active compounds exceeds the sum of the activity of each active compound alone. The term “synergistically effective amount,” as used herein, means and includes an amount of two or more active compounds that provides a synergistic effect defined above.

All terms used herein are intended to have their ordinary meaning unless otherwise provided. The phrases “pharmaceutically acceptable,” “physiologically acceptable,” or “physiologically compatible” is used interchangeably with “cosmetically acceptable,” “topically acceptable” and “dermatologically acceptable” and is intended to mean that a particular component is generally regarding as safe and non-toxic for application to a human integument (e.g., skin) at the levels employed.

Disclosed herein is a composition for improving skin health of a subject, and particularly, skin health of a subject seeking to limit or reduce the effects of age on the skin. While past attempts at reducing or limiting the effects of age on skin have employed topical compositions to the skin, the instant disclosure shows the surprising and unexpected result that systematic (e.g., oral) administration of the disclosed composition can achieve a substantial reversal of the effects of aging on the skin.

In certain embodiments, the disclosed compositions included an effective amount of collagen and elastin. In certain embodiments, collagen and elastin are present at a collagen to elastin ratio of about 10:1. Ratios used herein are weight:weight unless otherwise indicated. According to certain further embodiments, collagen and elastin are present at a collagen to elastin ratio of from about 10:1 to about 30:1 (w:w). In yet further embodiments, collagen and elastin are present at a collagen to elastin ratio of about 25:1 (w:w).

In certain embodiments, the collagen is low molecular weight collagen. For example, in certain embodiments the low molecular weight collagen has been hydrolyzed and is thus distinct from the form of collagen occurring in nature. In exemplary implementations of these embodiments, the average molecular weight of the collagen ranges from about 1500 Da to about 6000 Da. According to certain further embodiments, the average molecular weight of the collagen ranges from about 2000 Da to about 5000 Da.

In certain embodiments, the elastin is low molecular weight elastin. For example, in certain embodiments the low molecular weight elastin has been hydrolyzed and is thus distinct from the form of elastin occurring in nature. In exemplary implementations, the average molecular weight of the elastin ranges from about 900 to about 2000 Da. In further embodiments, the average molecular weight of the elastin ranges from about 1200 to about 1600 Da. In still further embodiments, the average molecular weight of the elastin is about 1400 Da.

According to certain embodiments, the composition further comprises a carrier. In certain embodiments, the carrier is a nutraceutically acceptable carrier. In further embodiments, the nutraceutically acceptable carrier is gel cap carrier. In still further embodiments, the gel cap carrier is a vegetal based gel cap carrier.

In certain embodiments, the composition is formulated such that a dose contains collagen ranging from about 1 to about 1000 mg (e.g., about 1 mg, about 5 mg, about 10 mg, about 15 mg, about 20 mg, about 25 mg, about 30 mg, about 35 mg, about 40 mg, about 45 mg, about 50 mg, about 75 mg, 100, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg, and the like, or any range or value therein).

In certain embodiments, the composition is formulated such that a dose contains collagen ranging from about 500 to about 13,500 mg (e.g., about 500 mg, about 1000 mg, about 1,500 mg, about 2,000 mg, about 2,500 mg, about 3,000 mg, about 3,500 mg, about 4,0000 mg, about 4,500 mg, about 5,000 mg, about 7,500 mg, 10,000, about 13,500 mg, and the like, or any range or value therein).

In certain embodiments, the composition is formulated such that a dose contains elastin ranging from about 1 to about 1000 mg (e.g., about 1 mg, about 5 mg, about 10 mg, about 15 mg, about 20 mg, about 25 mg, about 30 mg, about 35 mg, about 40 mg, about 45 mg, about 50 mg, about 75 mg, 100, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg, and the like, or any range or value therein).

In certain embodiments, the composition is formulated such that a dose contains elastin ranging from about 500 to about 13,500 mg (e.g., about 500 mg, about 1000 mg, about 1,500 mg, about 2,000 mg, about 2,500 mg, about 3,000 mg, about 3,500 mg, about 4,0000 mg, about 4,500 mg, about 5,000 mg, about 7,500 mg, 10,000, about 13,500 mg, and the like, or any range or value therein).

In certain embodiments, the disclosed composition further comprises one or more additional skincare ingredients. In certain embodiments, the one or more additional skincare ingredients is hyaluronic acid. In certain embodiments, the hyaluronic acid comprises from about 2 to about 5% (w/w) of the composition. In further embodiments, the hyaluronic acid comprises from about 3 to about 4% (w/w) of the composition. In yet further embodiments, hyaluronic acid comprises 3.6% (w/w) of the composition.

In certain embodiments, the composition is formulated such that a dose contains hyaluronic acid ranging from about 1 to about 1000 mg (e.g., about 1 mg, about 5 mg, about 10 mg, about 15 mg, about 20 mg, about 25 mg, about 30 mg, about 35 mg, about 40 mg, about 45 mg, about 50 mg, about 75 mg, 100, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg, and the like, or any range or value therein. In certain embodiments, the composition is formulated such that a dose contains about 100 mg of hyaluronic acid.

According to still further embodiments, one or more additional skincare ingredients is vitamin C. In certain embodiments, the composition is formulated such that a dose contains vitamin C ranging from about 1 to about 1000 mg (e.g., about 1 mg, about 5 mg, about 10 mg, about 15 mg, about 20 mg, about 25 mg, about 30 mg, about 35 mg, about 40 mg, about 45 mg, about 50 mg, about 75 mg, 100, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg, and the like, or any range or value therein. In certain embodiments, the composition is formulated such that a dose contains about 100 mg of vitamin C.

According to still further embodiments, one or more additional skincare ingredients is vitamin C. In certain embodiments, the vitamin C comprises from about 1 to about 3% (w/w) of the composition. In further embodiments, comprises 2.2% (w/w) of the composition. A person having skill in the art will appreciate that further additional skincare ingredients are possible.

Further disclosed herein is a method of improving skin health of a subject comprising administering to the subject a composition disclosed herein. In exemplary implementations, the composition is administered to the subject in an oral dosage form. In certain embodiments, improving skin health produces a reduction or reversal of the appearance of skin aging in a subject.

According to certain embodiments, improved skin health is measured by one or more of a reduction in periocular fine lines, periocular wrinkles, and/or global wrinkles. According to certain further embodiments, improved skin health is measured by one or more of an increase in skin firmness, radiance, smoothness, pinch recoil, extensibility, resiliency, pure elasticity, and/or bio-elasticity.

In certain implementations, wherein administration of collagen and elastin produces a synergistic improvement in skin health relative to the administration of a comparable dose of collagen or elastin alone.

In certain embodiments, the subject is 30 years of age, or older. In further embodiments, the subject is about 40 years of age, or older. In still further embodiments, the subject is 50 years of age, or older. In yet further embodiments, the subject is 60 years of age, or older.

According to certain embodiments, the subject is a woman. In certain implementations, the woman is perimenopausal. In further implementation, the woman is menopausal. In yet further implementations, the woman is post-menopausal.

According to certain embodiments of the disclosed method, the composition is administered repeatedly at predetermined intervals. In certain embodiments, the composition is administered daily. In certain implementations, the composition is administered daily for from about 1 week to about 26 weeks. In further embodiments, the composition is administered daily for from about 3 weeks to about 18 weeks. In yet further embodiments, the composition is administered daily for from about 6 weeks to about 12 weeks. In yet further embodiments, the composition is administered daily for an indefinite period of time.

According to certain embodiments, the composition is administered in conjunction with one more skincare care regimen. In certain implementations, the one or more skincare regimen comprises a daily application of one or more topical skincare products.

Further disclosed herein is a nutritional supplement for enhancing skin health comprising a composition disclosed herein. In certain embodiments, the nutritional supplement is a dietary supplement. In certain implementations, the dietary supplement is powder or a capsule. In further implementations, the nutritional supplement is a functional food. In exemplary implementations of these embodiments, the functional food is a beverage, nutrition bar, yoghurt, or cereal.

The formulations can also be prepared with liposomes, micelles and microspheres. Liposomes are microscopic vesicles that have a lipid wall that comprises a lipid bilayer and, in the present context, encapsulate one or more components of the formulations. The liposomal preparations of the present invention include cationic (positively charged), anionic (negatively charged), and neutral preparations. Cationic liposomes are readily available. For example, N [1-2,3-dioleyloxy) propyl]-N, N, N-triethyl-ammonium (DOTMA) liposomes are available under the tradename Lipofectin® (GIBCO BRL, Grand Island, NY). Similarly, anionic and neutral liposomes are also available, for example, from Avanti Polar Lipids (Birmingham, AL) or can be easily prepared using readily available materials. Such materials include phosphatidylcholine, cholesterol, phosphatidyl ethanolamine, dioleoylphosphatidylcholine (DOPC), dioleoylphosphatidylglycerol (DOPG) and dioleoylphosphatidyl ethanolamine (DOPE), among others. These materials can also be mixed with DOTMA in appropriate proportions. Methods for preparing liposomes using these materials are well known in the art.

In the art, micelles are known to comprise surfactant molecules arranged such that their polar head groups form an outer spherical shell, while the hydrophobic hydrocarbon chains are oriented towards the center of the sphere, forming a core. The micelles are formed in an aqueous solution containing surfactant in a concentration high enough for the micelles to occur naturally. Useful surfactants to form micelles include, but are not limited to, potassium laurate, sodium octane sulfonate, sodium decano sulfonate, sodium dodecane sulfonate, sodium lauryl sulfate, sodium docusate, decyltrimethylammonium bromide, dodecyltrimethylammonium bromide, tetradecyltrimethylammonium bromide, tetradecyltrimethylammonium chloride, dodecylammonium chloride, polyoxyl-8-dodecyl ether, polyoxyl-12-dodecyl ether, nonoxynol 10 and nonoxynol 30.

Similarly, microspheres may be incorporated in the present formulations. Like the liposomes and micelles, the microspheres essentially encapsulate one or more components of the present formulations. Generally, although not necessarily, they are formed from lipids, preferably charged lipids such as phospholipids. The preparation of lipid microspheres is well known in the art and is described in the relevant texts and literature.

In further embodiments, the instantly disclosed composition may be in the form of beadlets. For the purpose of this patent application, a “beadlet” refers to a small, generally spherical or near-spherical granule, which consists of a core material encapsulated by one or more layers (e.g., polymer layers). These layers may serve various functions, including but not limited to protection, controlled release, and stability enhancement of the core material. The encapsulating layers may comprise materials such as polymers, lipids, gels, carbohydrates, or other suitable encapsulating agents. The beadlet structure is designed to ensure targeted delivery, improve handling characteristics, enhance shelf life, or modulate the release profile of the disclosed composition. Beadlets may vary in size, typically ranging from a few micrometers to several millimeters in diameter.

In certain embodiments, the disclosed composition is in the form of a gummy. As used herein, the term “gummy” or “gummies” refers to a chewable, jelly-like substance primarily used for the oral delivery of dietary supplements. Gummies are characterized by their solid, yet soft and elastic texture, which is typically achieved through the use of gelatin, pectin, agar, or other similar gelling agents. They are often sweetened and flavored to enhance palatability and may contain natural or artificial colorants to improve visual appeal. The composition of gummies can be tailored to cater to specific dietary needs (e.g., vegan, sugar-free, gluten-free) and target audiences (e.g., children, adults, athletes). The gummies are formulated to ensure stability, controlled release of active ingredients, and ease of consumption, providing an alternative to traditional forms of dietary supplements such as pills or capsules. This definition encompasses gummies of various shapes, sizes, textures, and compositions, as long as they serve the primary function of delivering the disclosed composition in a chewable form.

In one aspect, methods of treating skin to improve the health and/or appearance thereof are provided comprising administering a collagen and elastin composition disclosed herein to a subject in need thereof at least once daily for a first period of time (e.g., a predetermined period of time), typically from 1-24 month, or from 2-24 months, or from 3-24 months, or from 4-24 months, or from 5-24 months, or from 6-monts, or from 7-months, or from 1-7 months, or from 2-7 months, or from 3-7 months, or from 4-7 months, or from 5-7 months, or 7 months, etc. The period of time may be “predetermined,” by which is meant that prior to beginning the regimen the user determines or is instructed (e.g., by written instruction accompanying the product or obtained electronically on a computer) to use the treatment modality (e.g., orally) daily for a fixed number of consecutive days. Alternatively, the period of time may be determined by the user's response and/or reaction to daily use of the first treatment modality (e.g., oral ingestion of the first composition). For example, the first period of time may begin on the first day of administration/application and end on the appearance of irritation and/or redness, or may end on when there are observable improvements and/or reductions in a sign of skin aging, or reduction in cellulitis or may end on the onset of an efficacy plateau, etc.

In some embodiments, the compositions of the present disclosure can be used with additional skin care compositions as part of a skin care regimen. For example, in facial treatment and care, users typically use multiple products for cleansing, toning, and treating the skin of the face. Accordingly, the first product comprises a first composition typically capable of providing a first benefit to a user, and the second product comprises a second composition typically capable of providing a second benefit to a user. In the present disclosure, it should be understood that at least one of the products of the regime includes the composition of the present disclosure, thereby providing the benefit of reducing signs of skin aging. In some embodiments, it should be understood by one skilled in the art, that while the first product and second product can independently provide any benefit known in the art of the particular care regimen, in each particular multi-product care regimen, the first product and second product may include different compositions and thus, provide different benefits to the user.

### Nutritional Supplements

The compositions of the disclosure may take the form of dietary supplements or may themselves be used in combination with dietary supplements, also referred to herein as food supplements.

Nutritional supplements may be found in many forms such as tablets, capsules, soft gels, gel caps, liquids, or powders. Some dietary supplements can help ensure an adequate dietary intake of essential nutrients; others may help reduce the risk of disease.

### Food Products

The compositions of the disclosure may take the form of a food product. Here, the term “food” is used in a broad sense and covers food and drink for humans as well as food and drink for animals (i.e. a feed). Preferably, the food product is suitable for, and designed for, human consumption.

The food may be in the form of a liquid, solid or suspension, depending on the use and/or the mode of application and/or the mode of administration.

When in the form of a food product, the composition may comprise or be used in conjunction with one or more of: a nutritionally acceptable carrier, a nutritionally acceptable diluent, a nutritionally acceptable excipient, a nutritionally acceptable adjuvant, a nutritionally active ingredient.

By way of example, the compositions of the disclosure may take the form of one of the following: A fruit juice; a beverage comprising whey protein: a health or herbal tea, a cocoa drink, a coffee drink, a yoghurt and/or a drinking yoghurt, a cheese, an ice cream, a desserts, a confectionery, a biscuit, a cake, cake mix or cake filling, a snack food, a fruit filling, a cake or doughnut icing, an instant bakery filling cream, a filling for cookies, a ready-to-use bakery filling, a reduced calorie filling, an adult nutritional beverage, an acidified soy/juice beverage, a nutritional or health bar, a beverage powder, a calcium fortified soy milk, or a calcium fortified coffee beverage.

### Food Ingredients

Compositions of the present disclosure may take the form of a food ingredient and/or feed ingredient.

As used herein the term “food ingredient” or “feed ingredient” includes a composition which is or can be added to functional foods or foodstuffs as a nutritional and/or health supplement for humans and animals.

The food ingredient may be in the form of a liquid, suspension or solid, depending on the use and/or the mode of application and/or the mode of administration.

### Functional Foods

Compositions of the disclosure may take the form of functional foods. As used herein, the term “functional food” means food which is capable of providing not only a nutritional effect, but is also capable of delivering a further beneficial effect to the consumer.

Accordingly, functional foods are ordinary foods that have components or ingredients (such as those described herein) incorporated into them that impart to the food a specific function—e.g. medical or physiological benefit—other than a purely nutritional effect.

Although there is no legal definition of a functional food, most of the parties with an interest in this area agree that they are foods marketed as having specific health effects beyond basic nutritional effects.

Some functional foods are nutraceuticals. Here, the term “nutraceutical” means a food which is capable of providing not only a nutritional effect and/or a taste satisfaction, but is also capable of delivering a therapeutic (or other beneficial) effect to the consumer. Nutraceuticals cross the traditional dividing lines between foods and medicine.

### Medical Foods

Compositions of the present disclosure may take the form of medical foods. By “medical food” it is meant a food which is formulated to be consumed or administered with or without the supervision of a physician and which is intended for a specific dietary management or condition for which distinctive nutritional requirements, based on recognized scientific principles, are established by medical evaluation.

While multiple embodiments are disclosed, still other embodiments of the disclosure will become apparent to those skilled in the art from the following detailed description, which shows and describes illustrative embodiments of the disclosed compositions, systems and methods. As will be realized, the disclosed compositions, systems and methods are capable of modifications in various obvious aspects, all without departing from the spirit and scope of the disclosure. Accordingly, the drawings and detailed description are to be regarded as illustrative in nature and not restrictive.

Various aspects and embodiments of the present invention are defined by the following numbered clauses:
1. A composition for improving skin health of a subject, the composition comprising an effective amount of collagen and elastin.
2. The composition of clause 1, wherein collagen and elastin are present at a collagen to elastin ratio of from about 10:1 to about 30:1 (w:w).
3. The composition of clause 2, wherein collagen and elastin are present at a collagen to elastin ratio of about 25:1.
4. The composition of any of clauses 1-3, wherein the collagen is low molecular weight collagen.
5. The composition of clause 4, wherein the average molecular weight of the collagen ranges from about 2000 to about 5000 Da.
6. The composition of any of clauses 1-5, wherein the elastin is low molecular weight elastin.
7. The composition of clause 6, wherein the average molecular weight of the elastin ranges from about 1300 to about 1500 Da.
8. The composition of any of clauses 1-7, wherein the composition further comprises a gel cap carrier.
9. The composition of clause 8, wherein the gel cap carrier is a vegetal based gel cap carrier.
10. The composition of clause any of clauses 1-9, further comprising hyaluronic acid.
11. The composition of clause 10, wherein the hyaluronic acid comprises from about 2 to about 5% (w/w) of the composition.
12. The composition of clause 11, wherein hyaluronic acid comprises from about 3 to about 4% (w/w) of the composition.
13. The composition of clause 12, wherein hyaluronic acid comprises 3.6% (w/w) of the composition.
14. The composition of any of clauses 1-13, further comprising vitamin C.
15. The composition of clause 14, wherein the vitamin C comprises from about 1 to about 3% (w/w) of the composition.
16. The composition of clause 15, wherein the vitamin C comprises 2.2% (w/w) of the composition.
17. The composition of any of clauses 1-16, wherein the composition is formulated for oral administration.
18. The composition of clause 17, wherein the composition is not formulated for topical administration.
19. A method for improving skin health of a subject comprising administering to the subject the composition of any of clauses 1-18.
20. The method of clause 19, wherein improving skin health produces a reduction or reversal of the appearance of skin aging in a subject.
21. The method of clause 20, wherein the subject is 30 years of age or older.
22. The method of clause 21, wherein the subject is a woman.
23. The method of clauses 19-22, wherein improved skin health is measured by one or more of a reduction in periocular fine lines, periocular wrinkles, and/or global wrinkles.
24. The method of clauses 19-23, wherein improved skin health is measured by one or more of an increase in skin firmness, radiance, smoothness, pinch recoil, extensibility, resiliency, pure elasticity, and/or bio-elasticity.
25. The method of clauses 19-24, wherein administration of collagen and elastin produces a synergistic improvement in skin health relative to the administration of a comparable dose of collagen or elastin alone.
26. The method of clauses 19-25, wherein the composition is administered repeatedly at predetermined intervals.
27. The method of clause 26, wherein the composition is administered daily.
28. The method of clause 27, wherein the composition is administered daily for from about 1 week to about 26 weeks.
29. The method of clause 28, wherein the composition is administered daily for from about 3 weeks to about 18 weeks.
30. The method of clause 29, the composition is administered daily for from about 6 weeks to about 12 weeks.
31. The method of clauses 19-30, wherein the composition is administered in conjunction with one more skincare care regimen.
32. The method of clause 31, wherein the one or more skincare regimen comprises a daily application of one or more topical skincare products.
33. A nutritional supplement for enhancing skin health comprising composition of clauses 1-18.
34. The nutritional supplement of any of clause 33, wherein the nutritional supplement is a dietary supplement.
35. The nutritional supplement of clause 34, wherein the dietary supplement is powder or a capsule.
36. The nutritional supplement any of clause 33, wherein the nutritional supplement is a functional food.
37. The nutritional supplement of clause 36, wherein the functional food is a beverage, nutrition bar, yoghurt, or cereal.
38. The method of clauses 19-32, wherein the composition is administered such that the subject receives a collagen dose from about 1,500 mg to about 3,000 mg.
39. The method of clauses 19-32, wherein the composition is administered such that the subject receives a collagen dose of about 2,500 mg.
40. The method of clauses 19-32, wherein the composition is administered such that the subject receives an elastin dose of from about 50 mg to about 300 mg.
41. The method of clauses 19-32, wherein the composition is administered such that the subject receives elastin dose of about 100 mg.

### EXAMPLES

The following examples are put forth so as to provide those of ordinary skill in the art with a complete disclosure and description of certain examples of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

### Experimental Example 1

### Sample

A total of 89 subjects completed study participation, with 43 subjects in cell 1 (Product A: Placebo) and 46 subjects in cell 2 (Product B: Collagen Supplement Powder). Subjects were assigned to 1 of 2 treatment cells in accordance with a predetermined randomization. Subjects were blinded to treatment assignment and were not made aware of receiving the placebo or active product. The composition of Product B: Collagen Supplement Powder is set forth in Table A below. The average molecular weight of collagen and elastin in Product B was 2 kD and 1.5 kD, respectively.

TABLE A
Product B

| | | |
|---|---|---|
| Collagen | 2500 mg | 90.50% |
| Elastin | 100 mg | 3.60% |
| Hyaluronic | 100 mg | 3.60% |
| Acid | | |
| Vitamin C | 60 mg | 2.20% |

No participants started a new health regimen (e.g., changed diet, changed exercise routine, sun exposure) during the intervention. At the beginning of the study, subjects agreed that they were “willing to refrain from changing diet during the study.” In addition, participants received a reminder to continue with their usual diet, exercise routine, and sun exposure.

A summary of subject disposition information is included in Table 1. The demographic information for the Per-Protocol (PP) population is presented in Table 2. For applicable parameters, the number of subjects in each category is listed with the percentage of total subjects in parentheses.

TABLE 1
Subject Disposition

| | | Cell 1: | Cell 2: |
|---|---|---|---|
| | All Subjects | Placebo | Collagen |
| Enrolled Subjects | 95 | 47 | 48 |
| ITT Population | 91 | 44 | 47 |
| PP Population (Completed Subjects) | 89 | 43 | 46 |
| Discontinued Subjects | 6 | 4 | 2 |

TABLE 2
Summary of Demographic Information - PP Population

| | PP Subjects | Cell 1: Placebo | Cell 2: Collagen |
|---|---|---|---|
| # of Subjects | 89 | 43 | 46 |

Age (years)

| | | | |
|---|---|---|---|
| Mean | 63.3 | 62.8 | 63.8 |
| Standard Deviation | 5.8 | 5.9 | 5.8 |
| Minimum | 47 | 49 | 47 |
| Median | 65 | 63.0 | 66 |
| Maximum | 71 | 71 | 71 |

| | | | | | |
|---|---|---|---|---|---|
| # of | | # of | | # of | |
| Sub. | % | Sub. | % | Sub. | % |

Sex

| | | | | | | |
|---|---|---|---|---|---|---|
| Female | 89 | 100 | 43 | 100 | 46 | 100 |

Ethnicity

| | | | | | | |
|---|---|---|---|---|---|---|
| Hispanic or Latino | 10 | 11.2 | 5 | 11.6 | 5 | 10.9 |
| Not Hisp. or Latino | 79 | 88.8 | 38 | 88.4 | 41 | 89.1 |

Race

| | | | | | | |
|---|---|---|---|---|---|---|
| Asian | 12 | 13.5 | 5 | 11.6 | 7 | 15.2 |
| Black or African | 1 | 1.1 | 1 | 2.3 | 0 | 0 |
| American | | | | | | |
| White or Caucasian | 76 | 85.4 | 37 | 86.0 | 39 | 84.8 |

Fitzpatrick Skin Time

| | | | | | | |
|---|---|---|---|---|---|---|
| I | 7 | 7.9 | 4 | 9.3 | 3 | 6.5 |
| II | 41 | 46.1 | 18 | 41.9 | 23 | 50.0 |
| III | 33 | 37.1 | 18 | 41.9 | 15 | 32.6 |
| IV | 8 | 9.0 | 3 | 7.0 | 5 | 10.9 |

Skin Type

| | | | | | | |
|---|---|---|---|---|---|---|
| Combination | 42 | 47.2 | 19 | 44.2 | 23 | 50.0 |
| Dry | 12 | 13.5 | 7 | 16.3 | 5 | 10.9 |
| Normal | 32 | 36.0 | 17 | 39.5 | 15 | 32.6 |
| Oily | 3 | 3.4 | 0 | 0 | 3 | 6.5 |

### Testing Procedure

During the course of the study, subjects consumed the assigned supplement once per day as directed. Additionally, all subjects could use a gentle skin cleanser and daily facial moisturizer with sunscreen broad spectrum SPF 15 as normally used. The following usage instructions were explained by clinic personnel:
Stir one scoop (2.6 g) of the test product into a hot or cold beverage and consume, once daily. May be taken at any time of the day, before or with food. Do not exceed this usage.
Wash your face with the provided cleanser as you normally would. You do not have to use the cleanser if you normally don't wash your face with anything except water.
Once daily, you may apply a sufficient amount of the provided moisturizer to the entire face. You do not have to apply the moisturizer if you don't need it. If you wish to apply the moisturizer, do not apply more than 1 time per day. Do not apply moisturizer within 24 hours before a clinic visit. If you apply more than once, you will have to reschedule your visit.
Store the products in a cool, dry place that is away from direct heat and light (including sunlight) and protected from contamination.

Subjects were provided with a calendar of study visits, written usage/study instructions, and a daily diary to record product usage and comments. Clinical evaluations were conducted at visit 1 (baseline), visit 2 (week 4), and visit 3 (week 8). Evaluation methods were performed as listed in the Measurement Techniques section. One week after visit 1, a clinic staff member called each subject to confirm each subject was consuming the assigned supplement and using the supporting materials as instructed.

### Measurement Techniques

Prior to the start of the study, prospective subjects were prescreened for eligibility criteria using an Institutional Review Board approved script. Women 45 to 71 years of age, with normal, oily, dry, or combination skin, were scheduled for eligibility screening at the clinic.

### Prescreening

At visit 1 (baseline), prospective subjects completed the informed consent process and signed an informed consent form. Prospective subjects who signed this initial paperwork were assigned a screening number and acclimated to ambient temperature and humidity conditions for at least 15 minutes prior to participating in evaluation procedures. During the course of the study, every effort was made to maintain applicable rooms at a temperature of 68-75° F. and relative humidity of 35%-65%. Clinic personnel and ensured that prospective subjects had a clean face with no makeup or topical products prior to participating in study procedures.

Prospective subjects were screened for study eligibility by the Fitzpatrick Type Skin Classification, Facial Skin Condition, and Topical Product Usage criteria.

### Fitzpatrick Type Skin Classification.

The Fitzpatrick skin classification is based on the skin's unprotected response to the first 30 to 45 minutes of sun exposure after a winter season without sun exposure. The categories of skin types are given in Table 3. Skin types I-IV were selected as eligible for the study.

TABLE 3

| | | |
|---|---|---|
| I | White; very fair; red or blonde | Always burns easily; never tans |
| | hair; blue eyes; freckles | |
| II | White; fair; red or blonde hair; | Always burns easily; tans |
| | blue, hazel, or green eyes | minimally |
| III | Cream white; fair with any eye | Burns moderately; tans gradually |
| | or hair color; very common | |
| IV | Brown; typical Mediterranean | Burns minimally; always tans well |
| | white skin | |
| V | Dark brown; mid-eastern skin | Rarely burns; tans profusely |
| | types, black hair, olive skin | |
| VI | Black; black hair, black eyes, | Never burns; deeply pigmented |
| | black skin | |

### Facial Skin Conditions

Clinically determined mild to moderate conditions (score of 3-6 according to a modified Griffiths scale ¹where 0=none and 9=severe) for the following parameters on the indicated locations:
Fine lines on periocular area and global face
Wrinkles on periocular area and global face
Lack of firmness (sagging) on the global face
Dullness/lack of radiance on the global face

### Topical Product Usage

Clinic personnel inspected and documented each candidate subject's topical products to ensure they do not contain antiaging ingredients.

### Initial Measurements

### Clinical Grading of Efficacy Parameters

Each subject was clinically graded for fine lines and wrinkles on the periocular area and global face; skin smoothness (visual) on the cheeks; and firmness (sagging) (visual), radiance, and overall appearance of skin condition (healthy) on the global face.

Additionally, triplicate pinch recoil measurements were performed on the left crow's feet area to assess skin elasticity/resiliency. ¹ Griffiths, C E, Wang T S, Hamilton T A, Voorhees J J, Ellis C N. A photonumeric scale for the assessment of cutaneous photodamage. Arch Dermatol. 1992; 128(3):347-351.

The following efficacy parameters were assessed at the indicated locations using a modified Griffiths 10-point scale according to the following numerical definitions (with half-point scores assigned as necessary to more accurately describe the skin condition):
0=none (best possible condition)
1 to 3=mild
4 to 6=moderate
7 to 9=severe (worst possible condition)
   The parameters in Table 4 were graded according to the listed scale anchors.

TABLE 4

| Parameter | Location(s) | 0= | 9= |
|---|---|---|---|
| Fine Lines | Periocular | None | Numerous and/or |
| | Global Face | | long fine lines |
| Wrinkles | Periocular | None | Numerous and/or |
| | Global Face | | long wrinkles |
| Skin Smoothness | Cheeks | Smooth, even- | Rough, uneven- |
| (Visual) | | looking skin | looking skin |
| | | texture, no | texture |
| | | roughness | |
| Firmness (Sagging- | Global Face | Lifted, tight- | Sagging, loose- |
| Visual) | | appearing skin | appearing skin |
| Radiance | Global Face | Radiant, | Dull/matte and/or |
| | | luminous, or | sallow skin |
| | | glowing | appearance |
| | | appearance | |
| Overall Appearance | Global Face | Excellent, healthy | Poor, unhealthy |
| of Skin Condition | | skin tone free | skin tone; |
| (Healthy) | | from skin | extensive skin |
| | | abnormalities | abnormalities |

Triplicate pinch recoil measurements were performed on the left crow's feet area. The skin was pinched between the forefinger and thumb and held for 2 seconds. Upon release, a stopwatch was used to record the amount of time it took for skin to return to its resting position. Each measurement was recorded to a hundredth of a second. A decrease in pinch recoil times indicates an improvement in skin “bounce-back” or elasticity/resiliency.

After the Clinical Grading of Efficacy Parameters measurements, each candidate subject's eligibility was reviewed, and qualified subjects continued with further measurements.

### Digital Imaging Using Portrait Photo Station

Clinic personnel ensured that subjects removed any jewelry from the areas to be photographed. Clinic personnel provided subjects with a black matte headband to keep hair away from the face and draped a black matte cloth over the subjects' clothing. Subjects were instructed to adopt neutral, relaxed, nonsmiling expressions with their eyes open, and were carefully positioned for each photograph.

Full-face digital images were taken from each subject (left, center, and right views) using the SGS Stephens, Inc. portrait photo station with a Canon Mark II 7D digital SLR camera and a Canon EF-S 60 mm f/2.8 macro lens under visible lighting mode. Unfiltered full-spectrum (white) light was provided using Comet studio strobes affixed to the photo station.

At each photography visit, Tiffen Grayscale color standards were photographed prior to beginning each day's photography. The CasMatch Color mire from Bear Medic Corporation and white, gray, and black chips from Pantone LLC color standard was included within each image.

### Antera 3D Imaging

Digital images were taken from each subject's left or right cheek (at the intersection of lines extending down from the corner of the eye and horizontally across the bottom of the nose), as determined by the grader, using Antera 3D® imaging. The field of view was 56 mm×56 mm with lateral resolution of 0.1 mm and vertical resolution 0.1 mm.

Antera 3D relies on multi-directional illumination and computer-aided reconstruction of the skin surface, illuminating the surface with LEDs of different wavelengths shining from different angles and using the differences between these images to reconstruct the surface in three dimensions.

### Cutometer Measurements

Duplicate measurements were taken from adjacent sites on each subject's right under-eye orbital bone using Cutometer MPA 580 to measure the viscoelastic properties (i.e., firmness and elasticity) of the skin.

The instrument applied a vacuum to a small area of the skin and measured the elastic response of the skin. The probe was kept at a 90° angle during the measurements. Negative pressure of 450 mbar was applied and released through a 2-mm probe. The measurement lasted for 20 seconds, during which there were 2 cycles of a 5-second on (vacuum) time and a 5-second off (skin release) time. The movement of the skin into and out of the probe was recorded during the application and release of suction. The amount of extensibility (RO, firmness), resiliency (R2), pure elasticity (R5) and biological elasticity (R7) were recorded.

The Cutometer trace was reviewed for proper shape before being saved. All subjects had suitable measurements at baseline.

### Subsequent Measurements

When subjects returned to the clinic for visit 2 (week 4) and visit 3 (week 8), subjects participated in the following procedures at each visit (unless otherwise indicated):
Clinic personnel recorded concomitant medications and questioned subjects regarding changes in their health. AEs were recorded if applicable.
Daily diaries were collected and reviewed for AEs and compliance. New diaries were distributed at week 4 and completed diaries were retained by the clinic.
Test and supporting material units were collected and visually inspected for compliance. Test material units were also weighed. Test and supporting material units were returned to subjects or new units were distributed as needed at week 4 and retained by the clinic at study completion.
Subjects acclimated for at least 15 minutes and then participated in the following procedures as previously described:
   Digital Imaging using Portrait Photo Station
   Antera 3D Imaging
   Clinical Grading of Efficacy Parameters
   Cutometer Measurements
At week 8, subjects completed a Sponsor-provided self-assessment questionnaire regarding skin improvements and product perception.

After study completion, Antera 3D images were analyzed for mean roughness (Ra) using Antera 3D CS software.

### Self-Assessment

At week 8 only, subjects completed a Sponsor-provided self-assessment questionnaire regarding skin improvements and product perception.

### Results

### Clinical Grading of Efficacy Parameters

TABLE 5
Fine Lines Periocular

| | Placebo | | | Collagen Supplement | | |
|---|---|---|---|---|---|---|
| | Baseline | Week 4 | Week 8 | Baseline | Week 4 | Week 8 |
| | Mean (SD) | Mean (SD) | Mean (SD) | Mean (SD) | Mean (SD) | Mean (SD) |
| Fine Lines | 3.95 (0.79) | 3.77 (0.86)* | 3.71 (0.85)+# | 4.16 (0.91) | 3.98 (0.98)* | 3.82 (1.00)+# |
| Periocular | | | | | | |

| | |
|---|---|
| Time | F(2, 174) = 45.25, p < .001 |
| Interaction | F(2, 174) = 1.83, p = 0.163 |
| Condition | F(2, 174) = 1717.61, p < .001 |

Interpretation: Lower scores indicate an improvement in periocular fine lines. Significant main effects for Time and Condition were found. Collagen Group improved significantly from Baseline to Week 4, Week 4 to Week 8, and Baseline to Week 8. The Collagen Group (0.34 difference from Baseline to Week 8) also outperformed the Placebo Group (0.24 difference).
Measure: Clinically graded on periocular area - area around the eye which may include features like eyelids, eyelashes, eyebrows, eye shape, skin texture.
*indicates significant differences from Baseline to Week 4
+indicates a significant difference between Baseline and Week 8
#indicates a significant difference between Week 4 and Week 8.

TABLE 6
Wrinkles Periocular

| | Placebo | | | Collagen Supplement | | |
|---|---|---|---|---|---|---|
| | Baseline | Week 4 | Week 8 | Baseline | Week 4 | Week 8 |
| | Mean (SD) | Mean (SD) | Mean (SD) | Mean (SD) | Mean (SD) | Mean (SD) |
| Wrinkles | 4.66 (0.64) | 4.62 (0.71) | 4.57 (0.73)+# | 4.87 (0.85) | 4.85 (0.85) | 4.77 (0.88)+# |
| Periocular | | | | | | |

| | |
|---|---|
| Time | F(2, 174) = 11.17, p < .001 |
| Interaction | F(2, 174) = 0.30, p = 0.741 |
| Condition | F(2, 174) = 3289.58, p < .001 |

Interpretation: Lower scores indicate an improvement in periocular wrinkles. Significant main effects for Time and Condition were found. Collagen Group had significant improvements from Baseline to Week 8 and Week 4 to Week 8. The Collagen Group (0.10 difference from Baseline to Week 8) outperformed the Placebo Group (0.09 difference).
Measure: Clinically graded on periocular area - area around the eye which may include features like eyelids, eyelashes, eyebrows, tear duct, eye shape, skin texture.
* indicates significant differences from Baseline to Week 4
+indicates a significant difference between Baseline and Week 8
#indicates a significant difference between Week 4 and Week 8.

TABLE 7
Fine Lines Global

| | Placebo | | | Collagen Supplement | | |
|---|---|---|---|---|---|---|
| | Baseline | Week 4 | Week 8 | Baseline | Week 4 | Week 8 |
| | Mean (SD) | Mean (SD) | Mean (SD) | Mean (SD) | Mean (SD) | Mean (SD) |
| Fine Lines | 4.38 (0.79) | 4.28 (0.82)* | 4.23 (0.82)+# | 4.34 (0.89) | 4.08 (0.94)* | 3.97 (0.87)+# |
| Global | | | | | | |

| | |
|---|---|
| Time | F(2, 174) = 44.83, p < .001 |
| Interaction | F(2, 174) = 7.94, p < 0.001 |
| Condition | F(2, 174) = 2216.26, p < .001 |

Interpretation: Lower scores indicate an improvement in global fine lines. Significant main effects for Time, Condition, and Interaction were evidenced. Collagen Group improved significantly from Baseline to Week 4, Week 4 to Week 8, and Baseline to Week 8. The Collagen Group (Baseline to Week 8 improvement = 0.37) significantly outperformed the Placebo Group (Baseline to Week 8 improvement = 0.15) over time (interaction).
*indicates significant differences from Baseline to Week 4
+indicates a significant difference between Baseline and Week 8
#indicates a significant difference between Week 4 and Week 8.

TABLE 8
Wrinkles Global

| | Placebo | | | Collagen Supplement | | |
|---|---|---|---|---|---|---|
| | Baseline | Week 4 | Week 8 | Baseline | Week 4 | Week 8 |
| | Mean (SD) | Mean (SD) | Mean (SD) | Mean (SD) | Mean (SD) | Mean (SD) |
| Wrinkles | 4.92 (0.68) | 4.87 (0.66)* | 4.85 (0.69)+# | 4.98 (0.61) | 4.97 (0.62)* | 4.95 (0.62)+# |
| Global | | | | | | |

| | |
|---|---|
| Time | F(2, 174) = 6.64, p = .002 |
| Interaction | F(2, 174) = 1.12, p = 0.33 |
| Condition | F(2, 174) = 5250.98, p < .001 |

Interpretation: Lower scores indicate an improvement in global wrinkles. Significant main effects for Time and Condition were found.
*indicates significant differences from Baseline to Week 4
+indicates a significant difference between Baseline and Week 8
#indicates a significant difference between Week 4 and Week 8.

TABLE 9
Firmness (sagging)

| | Placebo | | | Collagen Supplement | | |
|---|---|---|---|---|---|---|
| | Baseline | Week 4 | Week 8 | Baseline | Week 4 | Week 8 |
| | Mean (SD) | Mean (SD) | Mean (SD) | Mean (SD) | Mean (SD) | Mean (SD) |
| Firmness | 5.07 (0.51) | 5.02 (0.49)* | 5.01 (0.51)+# | 4.99 (0.52) | 4.97 (0.56)* | 4.83 (0.62)+# |

| | |
|---|---|
| Time | F(2, 174) = 16.37, p < .001 |
| Interaction | F(2, 174) = 6.05, p = 0.003 |
| Condition | F(2, 174) = 6617.40, p < .001 |

Interpretation: Lower scores indicate an improvement in firmness. Significant main effects for Time, Condition, and Interaction were evidenced. Collagen Group improved significantly from Baseline to Week 4, Week 4 to Week 8, and Baseline to Week 8. Collagen Group improved (Improvement = 0.16) significantly over time compared to the Placebo Group (0.06).
*indicates significant differences from Baseline to Week 4
+indicates a significant difference between Baseline and Week 8
#indicates a significant difference between Week 4 and Week 8.

TABLE 10
Radiance

| | Placebo | | | Collagen Supplement | | |
|---|---|---|---|---|---|---|
| | Baseline | Week 4 | Week 8 | Baseline | Week 4 | Week 8 |
| | Mean (SD) | Mean (SD) | Mean (SD) | Mean (SD) | Mean (SD) | Mean (SD) |
| Radiance | 4.94 (0.47) | 4.90 (0.49)* | 4.78 (0.54)+# | 4.82 (0.48) | 4.73 (0.53)* | 4.60 (0.53)+# |

| | |
|---|---|
| Time | F(2, 174) = 20.19, p < .001 |
| Interaction | F(2, 174) = 0.44, p = 0.65 |
| Condition | F(2, 174) = 8846.71, p < .001 |

Interpretation: Lower scores indicate an improvement in radiance. Collagen Group improved significantly from Baseline to Week 4, Week 4 to Week 8, and Baseline to Week 8. The Collagen Group (0.22 difference from Baseline to Week 8) outperformed the Placebo Group (0.16 difference).
*indicates significant differences from Baseline to Week 4
+indicates a significant difference between Baseline and Week 8
#indicates a significant difference between Week 4 and Week 8.

TABLE 11
Overall Appearance of Skin Condition (healthy)

| | Placebo | | | Collagen Supplement | | |
|---|---|---|---|---|---|---|
| | Baseline | Week 4 | Week 8 | Baseline | Week 4 | Week 8 |
| | Mean (SD) | Mean (SD) | Mean (SD) | Mean (SD) | Mean (SD) | Mean (SD) |
| Overall | 5.02 (0.55) | 5.00 (0.52) | 5.01 (0.56)+# | 5.03 (0.61) | 5.02 (0.61) | 4.84 (0.59)+# |
| Appearance | | | | | | |

| | |
|---|---|
| Time | F(2, 174) = 20.19, p < .001 |
| Interaction | F(2, 174) = 0.44, p = 0.65 |
| Condition | F(2, 174) = 8846.71, p < .001 |

Interpretation: Lower scores indicate an improvement in overall skin appearance. Collagen Group improved significantly from Week 4 to Week 8, and Baseline to Week 8. Collagen Group improved (Improvement = 0.19) significantly over time compared to the Placebo Group (0.01).
* indicates significant differences from Baseline to Week 4
+indicates a significant difference between Baseline and Week 8
#indicates a significant difference between Week 4 and Week 8.

### Cutometer Measurements

TABLE 12
Pinch Recoil

| | Placebo | | | Collagen Supplement | | |
|---|---|---|---|---|---|---|
| | Baseline | Week 4 | Week 8 | Baseline | Week 4 | Week 8 |
| | Mean (SD) | Mean (SD) | Mean (SD) | Mean (SD) | Mean (SD) | Mean (SD) |
| Pinch | 1.42 (0.34) | 1.47 (0.32)* | 1.48 (0.32)+ | 1.40 (0.30) | 1.38 (0.23)* | 1.43 (0.30)+ |
| Recoil | | | | | | |

| | |
|---|---|
| Time | F(2, 174) = 4.47, p = .013 |
| Interaction | F(2, 174) = 1.91, p = 0.151 |
| Condition | F(2, 174) = 2204.97, p < .001 |

Interpretation: Lower scores indicate an improvement in smoothness. Collagen Group improved significantly from Baseline to Week 4, Week 4 to Week 8, and Baseline to Week 8. The Collagen Group (Baseline to Week 8 improvement = 0.25) significantly outperformed the Placebo Group (Baseline to Week 8 improvement = 0.16) over time (interaction).
*indicates significant differences from Baseline to Week 4
+indicates a significant difference between Baseline and Week 8
# indicates a significant difference between Week 4 and Week 8.

TABLE 13
Extensibility

| | Placebo | | | Collagen Supplement | | |
|---|---|---|---|---|---|---|
| | Baseline | Week 4 | Week 8 | Baseline | Week 4 | Week 8 |
| | Mean (SD) | Mean (SD) | Mean (SD) | Mean (SD) | Mean (SD) | Mean (SD) |
| Extensibility | 0.31 (0.08) | 0.31 (0.07) | 0.33 (0.08)+# | 0.30 (0.06) | 0.30 (0.07) | 0.34 (0.06)+# |

| | |
|---|---|
| Time | F(2, 174) = 6.62, p = .002 |
| Interaction | F(2, 174) = 0.76, p = 0.471 |
| Condition | F(2, 174) = 2902.25, p < .001 |

Interpretation: Higher scores indicate an improvement in extensibility. Collagen Group had significant improvements from Baseline to Week 8 and Week 4 to Week 8. The Collagen Group (0.04 difference from Baseline to Week 8) outperformed the Placebo Group (0.02 difference).
* indicates significant differences from Baseline to Week 4
+indicates a significant difference between Baseline and Week 8
#indicates a significant difference between Week 4 and Week 8.

TABLE 14
Resiliency

| | Placebo | | | Collagen Supplement | | |
|---|---|---|---|---|---|---|
| | Baseline | Week 4 | Week 8 | Baseline | Week 4 | Week 8 |
| | Mean (SD) | Mean (SD) | Mean (SD) | Mean (SD) | Mean (SD) | Mean (SD) |
| Resiliency | 0.55 (0.12) | 0.55 (0.10) | 0.54 (0.09) | 0.53 (0.10) | 0.56 (0.09) | 0.54 (0.09) |

| | |
|---|---|
| Time | F(2, 174) = 1.17, p = .31 |
| Interaction | F(2, 174) = 0.76, p = 0.47 |
| Condition | F(2, 174) = 4137.36, p < .001 |

Interpretation: Lower scores indicate an improvement in resiliency. A significant Interaction was evidenced.
* indicates significant differences from Baseline to Week 4
+ indicates a significant difference between Baseline and Week 8
# indicates a significant difference between Week 4 and Week 8.

TABLE 15
Pure Elasticity

| | Placebo | | | Collagen Supplement | | |
|---|---|---|---|---|---|---|
| | Baseline | Week 4 | Week 8 | Baseline | Week 4 | Week 8 |
| | Mean (SD) | Mean (SD) | Mean (SD) | Mean (SD) | Mean (SD) | Mean (SD) |
| Pure | 0.50 (0.15) | 0.50 (0.13) | 0.42 (0.11)+# | 0.49 (0.14) | 0.50 (0.14) | 0.40 (0.08)+# |
| Elasticity | | | | | | |

| | |
|---|---|
| Time | F(2, 174) = 20.03, p < .001 |
| Interaction | F(2, 174) = 0.43, p = 0.653 |
| Condition | F(2, 174) = 2135.85, p < .001 |

Interpretation: Lower scores indicate an improvement in pure elasticity. Collagen Group improved significantly from Week 4 to Week 8, and Baseline to Week 8. The Collagen Group (0.09 difference from Baseline to Week 8) outperformed the Placebo Group (0.08 difference) in Interaction.
* indicates significant differences from Baseline to Week 4
+indicates a significant difference between Baseline and Week 8
#indicates a significant difference between Week 4 and Week 8.

TABLE 16
Biological Elasticity

| | Placebo | | | Collagen Supplement | | |
|---|---|---|---|---|---|---|
| | Baseline | Week 4 | Week 8 | Baseline | Week 4 | Week 8 |
| | Mean (SD) | Mean (SD) | Mean (SD) | Mean (SD) | Mean (SD) | Mean (SD) |
| Pure | 0.26 (0.06) | 0.25 (0.05) | 0.22 (0.05)+# | 0.25 (0.06) | 0.25 (0.05) | 0.22 (0.04)+# |
| Elasticity | | | | | | |

| | |
|---|---|
| Time | F(2, 174) = 18.74, p < .001 |
| Interaction | F(2, 174) = 0.20, p = 0.823 |
| Condition | F(2, 174) = 2925.87, p < .001 |

Interpretation: Lower scores indicate an improvement in biological elasticity. Significant main effects for Time and Condition were found.
* indicates significant differences from Baseline to Week 4
+indicates a significant difference between Baseline and Week 8
#indicates a significant difference between Week 4 and Week 8.

### Antera 3D Image Analysis Measurements

TABLE 17
Smoothness

| | Placebo | | | Collagen Supplement | | |
|---|---|---|---|---|---|---|
| | Baseline | Week 4 | Week 8 | Baseline | Week 4 | Week 8 |
| | Mean (SD) | Mean (SD) | Mean (SD) | Mean (SD) | Mean (SD) | Mean (SD) |
| Smoothness | 4.76 (0.48) | 4.58 (0.56)* | 4.60 (0.54)+ | 4.75 (0.49) | 4.61 (0.52)* | 4.50 (0.55)+ |

| | |
|---|---|
| Time | F(2, 174) = 29.02, p < .001 |
| Interaction | F(2, 174) = 3.06, p = 0.049 |
| Condition | F(2, 174) = 5725.61, p < .001 |

Interpretation: Lower scores indicate an improvement in smoothness. Collagen Group improved significantly from Baseline to Week 4, Week 4 to Week 8, and Baseline to Week 8. The Collagen Group (Baseline to Week 8 improvement = 0.25) significantly outperformed the Placebo Group (Baseline to Week 8 improvement = 0.16) over time (interaction).
*indicates significant differences from Baseline to Week 4
+indicates a significant difference between Baseline and Week 8
# indicates a significant difference between Week 4 and Week 8.

### Quantitative Results Summary

TABLE 18
Percent Improvement

| | Placebo | | | | | | Collagen | | | | |
|---|---|---|---|---|---|---|---|---|---|---|---|
| | % Change | | % Change | | % Change | | % Change | | % Change | % Change | |
| | BL to | | BL to | | Week 4 to | | BL to | | BL to | Week 4 to | |
| | Week 4 | | Week 8 | | Week 8 | | Week 4 | | Week 8 | Week 8 | |
| Fine Lines | 16 | (37%) | 21 | (49%) | 7 | (16%) | 16 | (35%) | 28 (61%) | 16 | (35%) |
| Periocular | | | | | | | | | | | |
| Wrinkles | 4 | (9%) | 7 | (16%) | 4 | (9%) | 2 | (4%) | 10 (22%) | 8 | (17%) |
| Periocular | | | | | | | | | | | |
| Fine Lines | 11 | (26%) | 13 | (30%) | 5 | (12%) | 24 | (52%) | 32 (70%) | 10 | (22%) |
| Global* | | | | | | | | | | | |
| Wrinkles | 4 | (9%) | 5 | (12%) | 2 | (5%) | 1 | (2%) | 3 (7%) | 2 | (4%) |
| Global | | | | | | | | | | | |
| Firmness* | 4 | (9%) | 5 | (12%) | 1 | (2%) | 3 | (7%) | 15 (33%) | 13 | (28%) |
| Radiance | 8 | (19%) | 17 | (40%) | 15 | (35%) | 9 | (20%) | 19 (41%) | 12 | (26%) |
| Overall Skin | 3 | (7%) | 4 | (9%) | 2 | (5%) | 1 | (2%) | 18 (39%) | 17 | (37%) |
| Appearance* | | | | | | | | | | | |
| Smoothness | 15 | (35%) | 15 | (35%) | 5 | (12%) | 14 | (30%) | 23 (50%) | 10 | (22%) |
| Pinch Recoil* | 14 | (33%) | 15 | (35%) | 19 | (44%) | 25 | (54%) | 25 (54%) | 24 | (52%) |
| Extensibility | 20 | (47%) | 18 | (42%) | 18 | (42%) | 22 | (48%) | 15 (33%) | 13 | (28%) |
| Resiliency | 21 | (49%) | 23 | (53%) | 25 | (58%) | 17 | (37%) | 19 (41%) | 26 | (57%) |
| Pure Elasticity | 19 | (44%) | 36 | (84%) | 32 | (74%) | 24 | (52%) | 34 (74%) | 37 | (80%) |
| Bio Elasticity | 21 | (49%) | 32 | (74%) | 31 | (72%) | 24 | (52%) | 34 (74%) | 32 | (70%) |

*Signifies a statistically significant difference via Chi Square analyses.

TABLE 19
Statistical Significance-Chi Squared Test

| | Placebo | | Collagen | | |
|---|---|---|---|---|---|
| | | Did Not | | Did Not | Statistic: Chi |
| | Improved | Improve | Improved | Improve | Square |
| Fine Lines | 13 | 30 | 32 | 14 | X2 = <0.001 |
| Global | | | | | |
| Firmness | 5 | 38 | 15 | 31 | X2 = 0.016 |
| Pinch recoil | 4 | 39 | 18 | 28 | X2 = 0.001 |

TABLE 20
Normality of the Data

| | Placebo | | | Collagen | | |
|---|---|---|---|---|---|---|
| | | Week | Week | | Week | Week |
| | Baseline | 4 | 8 | Baseline | 4 | 8 |
| Fine Lines | .229* | .161* | .141* | .153* | .168* | .145* |
| Periocular | | | | | | |
| Wrinkles | .195* | .222* | .176* | .132* | .143* | .130 |
| Periocular | | | | | | |
| Fine Lines | .156* | .138* | .136 | .178* | .150* | .152* |
| Global | | | | | | |
| Wrinkles | .160* | .161* | .145* | .174* | .181* | .172* |
| Global | | | | | | |
| Firmness | .271* | .264* | .255* | .227* | .243* | .198* |
| Radiance | .251* | .212* | .211* | .235* | .211* | .188* |
| Overall Skin | .291* | .271* | .273* | .265* | .273* | .291* |
| Appearance | | | | | | |
| Smoothness | .227* | .231* | .228* | .214* | .282* | .287* |
| Pinch Recoil | .077 | .091 | .091 | .108 | .105 | .086 |
| Extensibility | .135 | .092 | .090 | .068 | .125 | .091 |
| Resiliency | .146* | .095 | .085 | .140* | .083 | .065 |
| Pure Elasticity | .135 | .139* | .106 | .149* | .131 | .094 |
| Bio Elasticity | .106 | .097 | .068 | .163* | .170* | .135* |

*Signifies the data is normal.

TABLE 21
Skewness of the Data

| | Placebo | | | Collagen | | |
|---|---|---|---|---|---|---|
| | | Week | Week | | Week | Week |
| | Baseline | 4 | 8 | Baseline | 4 | 8 |
| Fine Lines | 0.057 | −.029 | .004 | .183 | −.223 | .195 |
| Periocular | | | | | | |
| Wrinkles | −.314 | −.640 | −.435 | −.251 | −.199 | −.110 |
| Periocular | | | | | | |
| Fine Lines | −.069 | .028 | .001 | −.196 | −.285 | −.229 |
| Global | | | | | | |
| Wrinkles | −.205 | −.046 | −.029 | −.295 | −.300 | −.214 |
| Global | | | | | | |
| Firmness | .446 | .242 | .104 | .000 | −.302 | −.047 |
| Radiance | −.278 | −.574 | −.135 | −.445 | −.662 | −.284 |
| Overall Skin | .052 | .156 | .525 | .50 | −1.098 | −1.313 |
| Appearance | | | | | | |
| Smoothness | .078 | −.249 | −.261 | .292 | .969 | 1.115 |
| Pinch Recoil | .013 | .499 | .782 | −.356 | −.061 | .087 |
| Extensibility | .649 | .229 | .348 | .169 | .632 | .133 |
| Resiliency | .591 | .945 | .310 | .794 | .283 | −.123 |
| Pure Elasticity | .812 | 1.151 | .554 | 1.136 | 1.045 | .840 |
| Bio Elasticity | .140 | .490 | .328 | .970 | .839 | .606 |

TABLE 22
Kurtosis of the Data

| | Placebo | | | Collagen | | |
|---|---|---|---|---|---|---|
| | | Week | Week | | Week | Week |
| | Baseline | 4 | 8 | Baseline | 4 | 8 |
| Fine Lines | 0.057 | −.029 | .004 | .183 | −.223 | .195 |
| Periocular | | | | | | |
| Wrinkles | −.314 | −.640 | −.435 | −.251 | −.199 | −.110 |
| Periocular | | | | | | |
| Fine Lines | −.069 | .028 | .001 | −.196 | −.285 | −.229 |
| Global | | | | | | |
| Wrinkles | −.205 | −.046 | −.029 | −.295 | −.300 | −.214 |
| Global | | | | | | |
| Firmness | .446 | .242 | .104 | .000 | −.302 | −.047 |
| Radiance | −.278 | −.574 | −.135 | −.445 | −.662 | −.284 |
| Overall Skin | .052 | .156 | .525 | .50 | −1.098 | −1.313 |
| Appearance | | | | | | |
| Smoothness | .078 | −.249 | −.261 | .292 | .969 | 1.115 |
| Pinch Recoil | .013 | .499 | .782 | −.356 | −.061 | .087 |
| Extensibility | .649 | .229 | .348 | .169 | .632 | .133 |
| Resiliency | .591 | .945 | .310 | .794 | .283 | −.123 |
| Pure Elasticity | .812 | 1.151 | .554 | 1.136 | 1.045 | .840 |
| Bio Elasticity | .140 | .490 | .328 | .970 | .839 | .606 |

### Self-Assessment Questionnaire Results

TABLE 23
Self-Assessment

| Inquiry | Placebo | Collagen |
|---|---|---|
| Did you find the product improved the overall | X | X |
| appearance of your skin? | | |
| After using the product, did you notice an | | |
| improvement in the following? | | |
| Fine Lines | — | — |
| Wrinkles | — | — |
| Texture | X | — |
| Radiance | — | — |
| Evenness of Skin Tone | — | — |
| Hydration/Moisture Level | — | X |
| Did you find the product improved any of the | | |
| following? | | |
| Forehead Lines | — | ◯ |
| Frown Lines (Between Eyebrows) | —\hat{ } | — |
| Eye Lines/Crow's Feet | — | — |
| Pigmentation/Discoloration | — | — |
| Undereye Circles | — | — |
| Nasolabial Folds (Smiles Lines) | — | — |
| Mouth Lines (Corners of Mouth) | — | — |
| Lip Lines (Above Upper Lip) | — | — |
| Marionette Lines (Chin) | — | — |
| Was the product easy to use every day? | X | X |
| Would you like to continue using this product? | X | X |

X Indicates that a statistically significantly greater proportion of subjects selected a favorable response (yes) compared. to the proportion of subjects who selected an unfavorable response (no).
◯ Indicates that a statistically significantly greater proportion of subjects selected an unfavorable response (no). compared to the proportion of subjects who selected a favorable response (yes).
— Indicates no statistically significant difference.
\hat{ }Indicates a statistically significant difference in favor of cell 1 (Product A: Placebo).

What is claimed is:

1. A composition for improving skin health of a subject, the composition comprising an effective amount of collagen and elastin, wherein collagen and elastin are present at a collagen to elastin ratio of from about 10:1 to about 30:1 (w:w), wherein the collagen is low molecular weight collagen having an average molecular weight from about 2000 Da to about 5000 Da, and wherein the elastin is low molecular weight elastin having an average molecular weight from about 1300 Da to about 1500 Da.

2. The composition of claim 1, wherein the collagen to elastin ratio is about 25:1 (w:w).

3. The composition of claim 1, further comprising hyaluronic acid.

4. The composition of claim 3, wherein the hyaluronic acid comprises from about 2% to about 5% (w/w) of the composition.

5. The composition of claim 4, wherein the hyaluronic acid comprises about 3.6% (w/w) of the composition.

6. The composition of claim 1, further comprising vitamin C.

7. The composition of claim 6, wherein the vitamin C comprises from about 1% to about 3% (w/w) of the composition.

8. The composition of claim 7, wherein the vitamin C comprises about 2.2% (w/w) of the composition.

9. The composition of claim 1, further comprising a gel cap carrier.

10. The composition of claim 1, wherein administration of the composition produces a synergistic improvement in skin health relative to administration of a comparable dose of collagen alone or elastin alone.

11. A method for improving skin health of a subject comprising orally administering to the subject a composition comprising collagen and elastin, wherein collagen and elastin are present at a collagen to elastin ratio of from about 10:1 to about 30:1 (w:w), wherein the collagen is low molecular weight collagen having an average molecular weight from about 2000 Da to about 5000 Da, wherein the elastin is low molecular weight elastin having an average molecular weight from about 1300 Da to about 1500 Da, and wherein the composition further comprises vitamin C in an amount from about 1% to about 3% (w/w) and hyaluronic acid in an amount from about 2% to about 5% (w/w).

12. The method of claim 11, wherein the hyaluronic acid comprises about 3.6% (w/w) of the composition and the vitamin C comprises about 2.2% (w/w) of the composition.

13. The method of claim 11, wherein the subject is 30 years of age or older and improving skin health produces a reduction or reversal of the appearance of skin aging in the subject.

14. The method of claim 11, wherein improved skin health is measured by one or more of a reduction in periocular fine lines, periocular wrinkles, and global wrinkles.

15. The method of claim 11, wherein improved skin health is measured by one or more of an increase in skin firmness, radiance, smoothness, pinch recoil, extensibility, resiliency, pure elasticity, and bio-elasticity.

16. The method of claim 11, wherein administration of the composition produces a synergistic improvement in skin health relative to the administration of a comparable dose of collagen or elastin alone.

17. The method of claim 11, wherein the composition is administered daily for from about 4 to about 8 weeks.

18. The method of claim 17, wherein the appearance of fine lines on the face of the subject decreases by from about 50% to about 70% relative to baseline.

19. The method of claim 17, wherein pinch recoil of the face of the subject increases by at least about 50% relative to baseline.

20. A nutritional supplement for enhancing skin health comprising: a composition comprising collagen and elastin present at a collagen to elastin ratio of from about 10:1 to about 30:1 (w:w), wherein the collagen is low molecular weight collagen having an average molecular weight from about 2000 Da to about 5000 Da and the elastin is low molecular weight elastin having an average molecular weight from about 1300 Da to about 1500 Da; hyaluronic acid comprising about 3.6% (w/w) of the supplement; and vitamin C comprising about 2.2% (w/w) of the supplement.
